(19) United States
(12) Patent Application Publication
(10) Pub. No.: US 2007/0265344 A1
(43) Pub. Date: Nov. 15, 2007

(54) Non-steroidal anti-inflammatory oral powder and liquid preparations for administration to animals

(75) Inventors: Michael Strobel, Northfield, MN (US); William Soderlund, St. Peter, MN (US); Patrick Soderlund, New Prague, MN (US)

Correspondence Address: CROWELL & MORING LLP;INTELLECTUAL PROPERTY GROUP, P.O. BOX 14300, WASHINGTON, DC, 20044-4300, US

(73) Assignee: Pharmaceutical Solutions, Inc.

(21) Appl. No.: 11/702,505

(22) Filed: Feb. 6, 2007

Related U.S. Application Data
(60) Provisional application No. 60/765,222, filed on Feb. 6, 2006.

(51) Int. Cl. A61K 31/195; A61P 29/02

(52) U.S. Cl. 514/570; 514/563

(57) ABSTRACT

A soluble formulation of a non-steroidal anti-inflammatory drug that can be orally administered to animals. The formulation includes a non-steroidal anti-inflammatory drug, a base of an amino acid, and a strong base. The formulation can be prepared in a dry or liquid form. The formulation can be dissolved in an animal's drinking water to provide an appropriate dosage of non-steroidal anti-inflammatory drug to the animal for treatment.

### FIELD OF THE INVENTION

The present invention relates to a palatable pharmaceutical composition for oral use in animals containing non-steroidal anti-inflammatory drugs, a base of an amino acid, and a strong base. More particularly, this invention relates to a palatable pharmaceutical composition of a propionic acid derivative, L-arginine or L-Lysine, and NaOH or another strong base.

### BACKGROUND

Non-steroidal anti-inflammatory drugs, usually abbreviated to NSAIDs, are drugs with analgesic, antipyretic and anti-inflammatory effects. The term “non-steroidal” is used to distinguish these drugs from steroids, which (amongst a broad range of other effects) have a similar eicosanoid-depressing, anti-inflammatory action. NSAIDS are unusual in that they are non-narcotic. NSAIDs are sometimes also referred to as non-steroidal anti-inflammatory agents/analgesics (NSAIAs). Members of this group of drugs include ibuprofen and ketoprofen.

Ibuprofen, i.e., 2-(4-Isobutylphenyl)propionic acid (said terms to be used interchangeably throughout this application), is a well-known anti-inflammatory drug having the following structure: 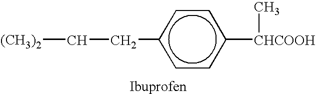
Ibuprofen is from the group of phenylpropionic acid derivatives that has shown itself to be effective in inhibiting prostaglandin synthesis in experiments with animals with inflammation. It is well known that ibuprofen is used to treat or reduce pain, inflammation, and fever.

Ibuprofen has an asymmetric carbon atom and is present as the racemate in the therapeutically-used form. It is known that for many pharmaceutically-active ingredients having one or more asymmetric carbon atoms that one enantiomeric form is often more effective or even much more effective than the other enantiomeric form. It is known that (R)-(−)-ibuprofen has substantially less pharmacological activity than (S)-(+)-ibuprofen. Nevertheless, most preparations contain the racemic mixture since the (R) enantiomer is converted to the active (S) form in-vivo. Hereafter, the term “ibuprofen” will be used to indicate any one of the (R) enantiomer, the (S) enantiomer, or the racemate.

Although ibuprofen has many advantages over other analgesics such as aspirin and acetaminophen, its solubility in water is very poor. For clarification, solubility is defined in this application as the amount of a substance that will dissolve in a given volume of solvent under specified conditions.

Animals will not ingest ibuprofen because of its bitter taste so ibuprofen is not considered palatable. Palatable as used in this application means acceptable or agreeable to drink or eat and, in the case of livestock, a formulation or compound that is voluntarily swallowed or taken in by the animal without any external means employed such as injection. Thus, it is very hard to administer ibuprofen in powder or pill form to an animal as the animal does not find the taste acceptable or agreeable and consequently will not ingest ibuprofen on its own accord.

U.S. Pat. No. 6,727,286 appears to describe a pharmaceutical composition for oral or injectable use containing ibuprofen and a basic amino acid, and more particularly, where the amino acid is arginine. All of the examples are injectable and the allegedly taught oral composition is bitter or not palatable. Moreover, solubility is lost at a ratio of 1:0.7 ibuprofen to L-Arginine. See Example 6 hereafter.

Similarly, U.S. Pat. No. 5,200,558 appears to describe enhanced analgesic effects of S (+) ibuprofen as salts of L and D amino acids, including arginine, in various dosage forms, including as an injectable solution. U.S. Pat. No. 4,279,926 appears to describe the use of basic amino acid salts of propionic acids for relieving pain and treating inflammatory conditions. Similarly, U.S. Pat. No. 5,463,117 appears to describe the preparation of salts of ibuprofen with basic amino acids. Finally, U.S. Pat. No. 6,005,005 appears to describe a liquid composition for oral use containing ibuprofen and arginine.

Similarly, U.S. Pat. Nos. 4,859,704 and 4,861,797 appear to describe the synthesis of alkali metal salts of ibuprofen for preparing a liquid ibuprofen formulation. U.S. Pat. No. 4,309,421 appears to describe water-soluble complexes of ibuprofen but with phospholipids for parenteral administration.

Other members of the propionic acid derivative family include ketoprofen, naproxen, flurbiprofen, fenoprofen, and oxaprozin. The pharmacodynamics properties of the propionic acid derivatives do not differ significantly. All are effective cyclooxygenase inhibitors, although there is considerable variation in their potency.

Ketoprofen, 2-(3-benzoylphenyl)-propionic acid (chemical formula C₁₆H₁₄O₃) is one of the propionic acid class of non-steroidal anti-inflammatory drug (NSAID) with analgesic and antipyretic effects. It acts by inhibiting the body's production of prostaglandin. The structural formula for ketoprofen is:

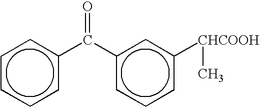

Although a number of references teach the use of ketoprofen, the ketoprofen compound itself is typically used with suitable organic and inorganic bases or oily solutions.

U.S. Pat. No. 6,069,172 (Bertini) describes a new use of the enantiomer (R)-ketoprofen and its salts with suitable organic and inorganic bases in the therapy of neutrophil-dependent diseases and phlogistic processes in a patient, and pharmaceutical preparations containing such compounds and useful for oral, parenteral or topical administration.

U.S. Pat. No. 5,665,384 (Courteille) describes stable, pharmaceutical ketoprofen salts for oral administration in oily solutions to avoid direct contact of acid forms of ketoprofen with the gastric or duodenal mucus membranes. Sodium, arginine, lysine and/or N-methylglucamine salts of ketoprofen are disclosed in solutions of polyoxyethyenatide vegetable oil, castor oil, esters of fatty acids and/or polyols. These oily solutions of ketoprofen may be administered orally in capsule form.

U.S. Pat. No. 6,995,190 (Strobel et al) describes an ingestible form of ketoprofen and an edible weak base for medication of animals. U.S. Pat. No. 6,995,190 does not disclose the use of a base of an amino acid to increase the bioavailability and solubility of the ketoprofen. Furthermore, U.S. Pat. No. 6,995,190 does not teach the use of a strong base.

Thus, certain non-injectable formulations of non-steroidal anti-inflammatory drugs, especially oral powders or liquid preparations, have been difficult to develop that are both soluble and palatable. The instant invention provides a soluble and palatable form of non-steroidal anti-inflammatory drugs that can easily be mixed into the animal's feed water and be provided to the animal without irritation. Moreover, the instant invention is an oral non-steroidal formulation that is palatable to the animal or livestock.

### SUMMARY

Embodiments of the instant invention include, but are not limited to, the following:

A dry solid non-steroidal anti-inflammatory drug formulation that can be administered to animals by dissolving it in the animal's drinking water. The formulation includes a non-steroidal anti-inflammatory drug, a base of an amino acid, and a strong base.

A dry solid ibuprofen formulation that can be administered to animals by dissolving it in the animal's drinking water. The formulation includes ibuprofen, a base of an amino acid, and a strong base.

A dry solid ketoprofen formulation that can be administered to animals by dissolving it in the animal's drinking water. The formulation includes ketoprofen, a base of an amino acid, and a strong base.

A liquid non-steroidal anti-inflammatory drug solution that can be administered to animals by adding it to the animal's drinking water. The formulation includes a non-steroidal anti-inflammatory drug, a base of an amino acid, and a strong base.

A liquid ibuprofen solution that can be administered to animals by adding it to the animal's drinking water. The formulation includes ibuprofen, a base of an amino acid, and a strong base.

A liquid ketoprofen solution that can be administered to animals by adding it to the animal's drinking water. The formulation includes ketoprofen, a base of an amino acid, and a strong base.

A method of administering a non-steroidal anti-inflammatory drug to animals for treatment. A dry solid or liquid ibuprofen formulation is prepared. The formulation includes a non-steroidal anti-inflammatory drug, a base of an amino acid, and a strong base. The formulation is then added to an animal's drinking water.

A method of administering ibuprofen to animals for treatment. A dry solid or liquid ibuprofen formulation is prepared. The formulation includes ibuprofen, a base of an amino acid, and a strong base. The formulation is then added to an animal's drinking water.

A method of administering ketoprofen to animals for treatment. A dry solid or liquid ketoprofen formulation is prepared. The formulation includes ketoprofen, a base of an amino acid, and a strong base. The formulation is then added to an animal's drinking water.

### DESCRIPTION

One embodiment of the invention is a soluble form of a non-steroidal anti-inflammatory drug that can be administered to animals. The soluble form is a mixture of a non-steroidal anti-inflammatory drug with a base of an amino acid and a strong base. The mixture can be prepared in either a powdered or liquid concentrate to be added to water to form a clear stock solution. Any form of a non-steroidal anti-inflammatory drug can be used to form the mixture such as pure non-steroidal anti-inflammatory drug or a salt of a non-steroidal anti-inflammatory drug. Any amino acid base may be used. Preferably, the amino acid base is L-Arginine or L-Lysine. The structures of the different amino acids whose bases can be used in the present invention are provided below. The amino acids are broken up into acids with hydrophobic side groups, acids with hydrophilic side groups, and acids with side groups in between hydrophilic and hydrophobic. Any strong base may be used. Examples of preferable strong bases include sodium hydroxide, di-sodium phosphate, tri-sodium phosphate, potassium hydroxide, and potassium phosphate.

TABLE 1
amino acids.
Amino acids with hydrophobic side groups

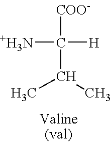

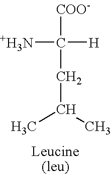

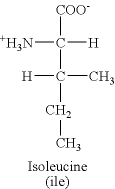

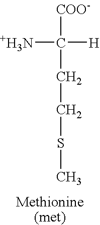

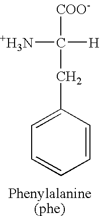

Amino acids with hydrophilic side groups

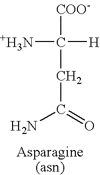

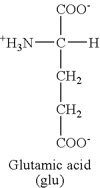

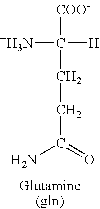

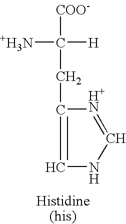

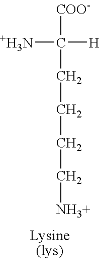

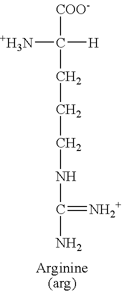

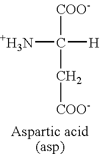

Amino acids that are in between

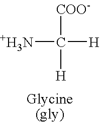

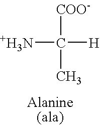

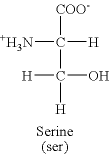

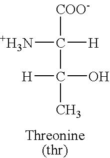

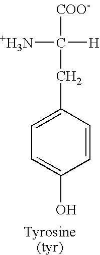

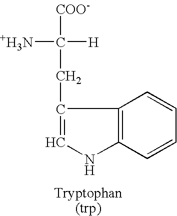

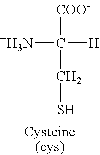

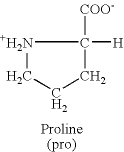

To enhance the palatability of the solution, flavorings and/or sweeteners can be added to the mixture. Any type of sweetener and/or flavoring can be added to the mixture to improve the palatability to the animals. Natural or artificial sweeteners and flavorings may be used. Examples of artificial sweeteners that can be used with the ibuprofen mixture include cyclohexyl-sulfamic acid, saccharin (o-benzosulfimide), and aspartame (i.e., 1-aspartyl-1-phenylalanine methyl ester) sold as Nutrasweet® artificial sweetener, and the like. If the animals would not be adversely affected by inclusion of sugar in the formulation, then sugar can be used to sweeten the solution. In actual practice, the sweetener and the flavoring are optional additives that may be used to enhance the palatable taste of the instant invention.

Preferably, the sweetener is an artificial sweetener. More preferably, the sweetener is sodium saccharin. Preferably, the flavoring used is cherry, strawberry or licorice. Of course, any other sweeteners or flavorings may also be used. Additionally, two or more flavorings may be included in a single mixture.

In one embodiment, a dry soluble form of a non-steroidal anti-inflammatory drug is formed. Preferably, ibuprofen or ketoprofen powder is used as the starting material. The mixing of the solid particulate ibuprofen or ketoprofen and the bases, with or without flavoring and sweetener, can be formed by any method using any equipment For example, small amounts of each ingredient can put into a plastic bag, typically made of polyethylene, or polyvinyl chloride-vinylidene chloride copolymer of Mylar® (a film form of polyethyleneterephthalate), and the like. The mixtures can be made in such bags by separately feeding solid particulate ibuprofen and solid particulate bases. Then the ingredients in the bag can be worked by hand to mix the particulates into an essentially homogeneously distributed mixture. The particulates can be mixed in large and small scale mixing equipment. They can be mixed in a household cake mixer, a large scale Banbury Mixer, large and small paddle mixers, and the like.

In another embodiment, a liquid formulation is prepared. In this embodiment, dry or liquid bases may be used. Additionally, a small amount of water can be used. The ingredients may be mixed together in any order by any method. Preferably, an ibuprofen or ketoprofen powder is used as the source of non-steroidal anti-inflammatory drug. Preferably, the ibuprofen or ketoprofen powder, the amino acid base, a strong base, and dionized water are all mixed together to form a liquid non-steroidal anti-inflammatory drug formulation. Sweeteners and flavorings can then be mixed into the liquid formulation.

The base of the amino acid increases the bioavailability of the non-steroidal anti-inflammatory drug to the animals and increases the solubility of the non-steroidal anti-inflammatory drug. In order to achieve the desired bioavailability and solubility, the weight ratio of the amino acid base to non-steroidal anti-inflammatory drug is between 0.25 and 1.0. Preferably the ratio is between 0.3 and 1.0. The weight ratio of the strong base to the non-steroidal anti-inflammatory drug is between 0.01 and 1.0. Preferably the ratio is between 0.3 and 1.0. The solid and liquid non-steroidal anti-inflammatory drug formulations of the present invention are very stable and can be stored for extended periods of time at room temperature 33-90° F. before being administered to animals.

The liquid or solid non-steroidal anti-inflammatory drug mixture can be easily administered to animals by mixing it in their drinking water. Preferably, the mixture is first dissolved in a small amount of water to form a stock solution. For example, one pint of the liquid non-steroidal anti-inflammatory drug mixture could be dissolved in one gallon of water to form a stock solution. For the dry solid formulation, the formulation could be added to one pint of water and then dissolved. The concentrated solution could then be dissolved in one gallon of water to form the stock solution as for the liquid formulation. The stock solution could then added to the drinking water. For example, one ounce of stock solution could used per gallon of drinking water.

The stock solution can be added directly to the animals' drinking water in a pail or stock tank or via a proportioner—Any proportioner can be used. The non-steroidal anti-inflammatory drug mixture when mixed with drinking water forms a stable, palatable preparation which is readily consumed by the animals. As administered to the animals, the pH of a stable, palatable ibuprofen preparation is typically 6.5 to 8.5 and preferably about 7.2.

The soluble non-steroidal anti-inflammatory drug preparation of the instant invention can be administered to any animal or human through their drinking water. The invention can be employed to treat all forms of domestic animals, such as livestock, e.g., pigs (including swine), beef and dairy cattle, horses, poultry (especially chickens and turkeys), sheep, and dogs, cats, and the like, as well as non-domestic animals such as deer, buffalo, and the like, that are kept in herds and fed from a controlled water supply. In particular, the ibuprofen preparation has been tested on swine, cattle and horses.

The ibuprofen preparation can be administered to the animal in any appropriate dosage as is known in the art of veterinary science. Preferably, the dosage is between 10 to 30 mg of ibuprofen per kg of animal weight. More preferably, the dosage is about 20 mg/kg. To achieve the desired dosage, one ounce of the stock solution can be added to each gallon of the animals drinking water until the desired amount of ibuprofen is administered to the animal over a 24 hour period.

### EXAMPLES

### Example 1

### Preparation of Liquid Ibuprofen Solution

For each gram of ibuprofen powder we used 0.3625 g L-Arginine base, 0.2258 g NaOH, 0.075 gm sodium Saccharin, 0.08125 ml Cherry Liquid qs and deionized water to make an aqueous ibuprofen solution which makes a 32% concentration liquid.

We prepared 5000 mL of the aqueous ibuprofen solution. We used 1,280 g of ibuprofen, 464 g of L-Arginine 289 g of Sodium hydroxide, 96 g sodium saccharin and 105 mL of Cherry concentrate. We added the ibuprofen, 1500 mL of water, the L-Arginine base and NaOH and mixed. Then we added the saccharin and cherry concentrate and red food coloring and qs to make 5000 mL total volume of aqueous Ibuprofen solution.

### Example 2

### Preparation of Dry Ibuprofen Mixture

We used 0.3625 g L-Arginine base, 1.0 g ibuprofen powder, 0.5 g of Di-sodium Phosphate 0.075 gm sodium saccharin and 0.02 g Cherry Powder. We blended the dry ingredients together in a V blender to form a dry uniform Ibuprofen mixture.

We measured out 110 g of the mixture and packaged in a water tight pouch. When administered to animals, 1 packet of mixture could be used per gallon of stock solution or 128 ounces of drinking water as fed to the animal.

### Example 3

### Test of Ibuprofen Formulation on Nursery Pigs

The product of example 1 was tested on 4000 nursery pigs which each weighed about 20 pounds. The aqueous ibuprofen solution was added to the water at a rate of 1 pint per gallon to form a stock solution. The stock solution was then administered through a proportioner at the rate of 1 oz of stock solution per gallon of drinking water for 14 consecutive days. No adverse side affects were noted during the course of treatment.

### Example 4

### Treatment of Pigs with Type A Influenza

The product used in example 3 was administered to pigs with Type A influenza (H1N1) at the first signs of disease for a period of 5 days. 2000 pigs were in the treatment group and 2000 pigs were in a control group. 50 pigs were randomly selected in each group and tempted daily for the 5 day treatment period. The average rectal temperature pretreatment was 104.8 in the medicated group and 104.7 in the control group. Table 1 shows the response to treatment. Table 2 shows the water consumption for each group.

TABLE 1
Rectal Temperature

| Group/Day | Treatment temp. (° F.) | Control temp. (° F.) |
|---|---|---|
| Before Treatment | 104.8 | 104.7 |
| 1 | 101.9 | 104.9 |
| 2 | 101.8 | 104.5 |
| 3 | 101.9 | 104.6 |
| 4 | 101.7 | 103.8 |
| 5 | 101.5 | 103.5 |

TABLE 2
Water Consumption

| | Treatment Group | Control Group |
|---|---|---|
| Day | (gal/day) | (gal/day) |
| 1 | 380 | 380 |
| 2 | 425 | 388 |
| 3 | 430 | 375 |
| 4 | 432 | 378 |
| 5 | 437 | 359 |

Feed consumption was normal in the treatment group and reduced by 42 percent in the control group. Mortality in the treatment group was 11 over the 4 weeks following the initiation of the treatment and 49 in the control group. Identical treatments other then the Ibuprofen were used in both groups.

### Example 5

### Treatment of Cattle with Pneumonia Due to Pasturella Multocida and Influenza

the product used in example 3 was used to cattle. The ibuprofen was administered via a stock tank along with the administration of injectable antibiotics. The ibuprofen was given at a rate of 10 mg per kg animal weight. There were 20 treatment and 20 control animals.

Both groups responded to treatment but the ibuprofen group had 25 percent greater feed and 20 percent better water consumption than the control groups and the average rectal temperature was 2.3 degrees lower.

### Example 6

### Solubility Test for Ibuprofen Oral Liquid w/L-arginine Alone

| | | Mix 1 gm ibuprofen | |
|---|---|---|---|
| | | vs X gm L arginine | |
| Ratio W/W ibuprofen | | Filtered/Dried | Oral |
| vs. L-Arginine | | Weight Powder | Palatability |
| | Observation | Recovered grams | Human |
| 1:1 | Clear | 0.00 | Bitter |
| 1:0.8 | Clear | 0.00 | Bitter |
| 1:0.7 | Clear/Turbid | 0.01 gm Ibuprophen | Very Bitter |
| 1:0.6 | Turbid/White | 0.3 gm | Very Bitter |
| 1:0.55 | White milky | 0.7 gm | Unpalatable |
| 1:0.50 | White Suspension | 1.0 gm | Unpalatable |
| 1:0.4 | White suspension | 1.0 gm | Unpalatable |

Conclusion: Complete solubility is achieved at Greater than 1 to 0.7 and reasonable solubility is greater than 1:0.6

### Example 7

### Palatability Test

L-arginine with NaOH mixed with ibuprofen 0.5:0.1:1 (Group 1) verses L-arginine/ibuprofen (Group 2) in the above example at 0.8:1 and 1:1

Water consumption when 72 grams of ibuprofen active is solubilized and mixed with 1 gallon of stock solution and administered at a rate of 1 oz per gallon of drinking water.

100 - 25 lb pigs 24 Hr water consumption

| | Gallons/24 hours |
|---|---|
| Group 1 | 32 |
| Group 2 | 11 |
| Control No Medication | 33 |

Conclusion: Significant improvement in oral palatability in the L-arginine/NaOH/ibuprofen group verses the L-Arginine/ibuprofen group

This experiment was repeated with KOH, Di-sodium Phosphate, and Tri-sodium phosphate in place of the NaOH with identical results.
We also tested with the NaOH alone and found a 25 percent reduction in water consumption but it was better than L-Arginine alone but worse than the mixture

Although specific examples have been provided where soluble non-steroidal anti-inflammatory drug mixtures of the instant invention have been tested on animals with certain diseases, it is to be understood that the non-steroidal anti-inflammatory drug mixtures could be used to treat animals with any ailments that non-steroidal anti-inflammatory drugs are known to be helpful or active in curing or treating. Examples include inflammation caused by disease or injury and fever of any origin. Additionally, the non-steroidal anti-inflammatory drug mixture can be provided to any animal.

What is claimed is:

1. A dry formulation comprising a non-steroidal anti-inflammatory drug, a base of an amino acid, and a strong base.

2. The formulation of claim 1, wherein the amino acid is lysine.

3. The formulation of claim 1, wherein the amino acid is arginine.

4. The formulation of claim 1, wherein the non-steroidal anti-inflammatory drug is ibuprofen.

5. The formulation of claim 1, wherein the non-steroidal anti-inflammatory drug is ketoprofen.

6. The formulation of claim 1, wherein the strong base is selected from the group consisting of sodium hydroxide, di-sodium phosphate, tri-sodium phosphate, potassium hydroxide, and potassium phosphate.

7. A palatable aqueous solution comprising a non-steroidal anti-inflammatory drug, a base of an amino acid, and a strong base.

8. The aqueous solution of claim 7, wherein the amino acid is lysine.

9. The aqueous solution of claim 7, wherein the amino acid is arginine.

10. The aqueous solution of claim 7, wherein the non-steroidal anti-inflammatory drug is ibuprofen.

11. The aqueous solution of claim 7, wherein the non-steroidal anti-inflammatory drug is ketoprofen.

12. The aqueous solution of claim 7, wherein the strong base is selected from the group consisting of sodium hydroxide, di-sodium phosphate, tri-sodium phosphate, potassium hydroxide, and potassium phosphate.

13. A method of administering a non-steroidal anti-inflammatory drug to animals comprising adding a liquid or solid mixture comprising a non-steroidal anti-inflammatory drug, a base of an amino acid and a strong base to the animals' drinking water.

14. The method of claim 13, wherein the amino acid is lysine.

15. The method of claim 13, wherein the amino acid is arginine.

16. The method of claim 13, wherein the non-steroidal anti-inflammatory drug is ibuprofen.

17. The method of claim 13, wherein the non-steroidal anti-inflammatory drug is ketoprofen.

18. The method of claim 13, wherein the strong base is selected from the group consisting of sodium hydroxide, di-sodium phosphate, tri-sodium phosphate, potassium hydroxide, and potassium phosphate.
